(19) United States
(12) Patent Application Publication
(10) Pub. No.: US 2015/0182546 A1
(43) Pub. Date: Jul. 2, 2015

(54) THERAPEUTIC AGENT FOR ENHANCING MITOCHONDRIAL FUNCTION

(71) Applicant: Mitochondrial Concepts LLC, Indianapolis, IN (US)

(72) Inventor: Charles A. Bonsett, Indianapolis, IN (US)

(73) Assignee: Mitochondrial Concepts LLC, Indianapolis, IN (US)

(21) Appl. No.: 14/382,791

(22) PCT Filed: Mar. 11, 2014
(86) PCT No.: PCT/US14/22931

§ 371 Date: Sep. 4, 2014

Related U.S. Application Data
(60) Provisional application No. 61/791,618, filed on Mar. 15, 2013.

(51) Int. Cl. A61K 31/7076; A61K 9/00

(52) U.S. Cl. CPC A61K 31/7076; A61K 9/0053; A61K 9/0019; A61K 9/0014

(57) ABSTRACT

Methods are provided for enhancing metabolic performance at the mitochondrial level. The methods include administering an adenylosuccinate in amounts sufficient to treat, in diseased tissue, symptoms and pathologies resulting from dysfunctional metabolism, i.e. subnormal production of adenosine triphosphate (ATP) and to enhance normal mitochondrial respiration in healthy tissue.

### CROSS-REFERENCE TO RELATED APPLICATIONS

This disclosure claims the benefit of priority to U.S. Provisional Patent Application 61/791,618 filed on 15 Mar. 2013, the disclosure of which is incorporated herein in its entirety.

### FIELD OF THE INVENTION

This invention relates generally to compounds and methods for treatment and prevention of diseases, and symptoms related to mitochondrial dysfunction.

### BACKGROUND

Mitochondrial diseases (inherited or otherwise) are characterized by inadequate energy production. The diseases can be a result of sex-linked inheritance, natural mutation, exposure to toxins, prescription medication, and/or ageing. Common to all mitochondrial diseases is the low production of adenosine triphosphate (ATP); this low ATP production can be a direct result or caused by diseases originating elsewhere, as for example the thyroid gland.

No cure exists for mitochondrial diseases. Standard medical therapies include administration of antioxidants (Vitamin E, Glutathione and Quinones i.e. Coenzyme Q10 (CoQ10) and Idebenone), “cocktails” of vitamins and co-factors (vitamins B1, B2, C, folic acid, L-carnitine, and creatine), and addressing abnormal changes in blood pH (which at the extreme can involve supplemental oxygen, intravenous fluid support, and mechanical ventilation). These therapies fail to alter the course of the underlying disease and may only off-set symptoms associated with low ATP production.

Moreover, the effectiveness of these mitochondrial disease therapies is neither well-proven nor generally accepted. Accordingly, patients with mitochondrial diseases are without clinically accepted therapies and suffer as a result.

### SUMMARY

Herein is disclosed a method of treating mitochondrial dysfunction in a subject in need, the method can include administering to the subject an effective amount of a composition comprising an adenylosuccinate (AS).

### DETAILED DESCRIPTION

This disclosure provides compounds and compositions that will enhance adenosine triphosphate (ATP) production in healthy mammal (e.g. human) mitochondria. This will enhance active transport, muscle contraction, and biosynthesis. Furthermore, this enhanced ATP production should both improve athletic performance and ameliorate the aging processes.

The compounds, compositions and methods described herein may be understood more readily by reference to the following detailed description and the examples provided. It is to be understood that this invention may not be limited to the specific components, articles, processes and/or conditions described, as these may, of course, vary. It is also to be understood that the terminology used herein is for the purpose of describing particular embodiments only and is not intended to be limiting.

Ranges may be expressed herein as from “about” or “approximately” one particular value or to “about” or “approximately” another particular value. When such a range is expressed, another embodiment includes from the one particular value or to the other particular value. Similarly, when values are expressed as approximations, by use of the antecedent “about,” it will be understood that the particular value forms another embodiment.

This disclosure also provides a method for treating pathophysiological consequences of mitochondrial respiratory chain deficiency resulting in a lack of ATP in a mammal (e.g. human). This treatment is comprised of administering to such mammal (e.g. human) an amount of an adenylosuccinate. The method can further include preventing the pathophysiological consequences of mitochondrial respiratory chain deficiencies that result in a lack of ATP. The efficacy of administering adenylosuccinate has been demonstrated through both in vitro and in vivo administration.

A first embodiment is a method of treating mitochondrial dysfunction in a subject in need. The method of treating the mitochondrial dysfunction includes administering to the subject an effective amount of a composition comprising an adenylosuccinate (AS).

In one example, the subject in need suffers from a mitochondrial respiratory chain deficiency disease. In one instance the mitochondrial respiratory deficiency disease is amyotrophic lateral sclerosis (ALS). In another example, the subject is need is an individual of advanced age (e.g. 70 years) wherein the subject has aging-related mitochondrial dysfunction.

The method consists of administering a composition containing adenylosuccinate in amounts sufficient to treat the symptoms and the pathologies resulting from a dysfunctional metabolism. It is hypothesized that a metabolic defect results from an inadequate production and reserve of adenosine triphosphate (ATP). The intracellular take-up of the adenylosuccinate is then followed by the adenylosuccinate crossing into cells and then into the mitochondria where adenylosuccinate drives the reaction adenylosuccinate→AMP→ADP→ATP strongly to the right, thus ameliorating the ATP deficiency. Preferably, the administration of the composition that includes the adenylosuccinate stops or arrests the degenerative effect of the lack of ATP.

The administered adenylosuccinate can be a salt selected from the group consisting of a lithium salt, a sodium salt, a potassium salt, an ammonium salt, a chelated alkali earth metal salt, a chelated transition metal salt, a cationic polymer, and a mixture thereof. Preferably the adenylosuccinate is a tetra-anion; that is, the adenylosuccinate is accompanied by a cation or cations that balance a 4-charge. In one particularly preferable instance, the adenylosuccinate is a tetrasodium adenylosuccinate (4SAS).

The administration of the adenylosuccinate may be accomplished via several approaches. It is possible to administer adenylosuccinate by means of a sub-cutaneous pump. However, oral administration of adenylosuccinate has proven efficacious in clinical trials. Alternatively, adenylosuccinate might be suitably packaged for timed release or trans-dermal application.

In one example the composition is an oral dosage; and the administration is by oral ingestion. The oral dosage can be a delayed release formulation that comprises the adenylosuccinate compounded with a delayed release agent. In another example, the composition is an intravenous dosage; and wherein the administration is sub-cutaneous. In still another example, the administration is transdermal and the composition is compounded and provided, for example, as a transdermal patch or a sublingual film. In still another example, the composition is an atomizable solution applicable by administration by inhaler.

Preferably, the administration of an effective amount treats the pathophysiological consequences of mitochondrial respiratory chain deficiency. In one example, the administration of an effective amount treats the pathophysiological consequences of pseudohypertrophic muscular dystrophy. In another example, the administration of an effective amount treats the pathophysiological consequences of amyotrophic lateral sclerosis. In still another example, the administration of an effective amount treats the pathophysiological consequences of advanced aging.

In one example, the administration of an effective amount includes the daily administration of about 2000 mg, about 1750 mg, about 1500, mg, about 1250 mg, about 1000 mg, about 750 mg, about 500 mg, about 400 mg, about 300 mg, about 250 mg, about 200 mg, about 200 mg, about 150 mg, about 100 mg, about 50 mg, about 40 mg, about 30 mg, about 20 mg, about 10 mg, about 5 mg, or about 1 mg. Preferably, the daily administration includes from about 1 gm to about 2000 mg, from about 1 mg, to about 1000 mg, from about 1 mg, to about 500 mg, from about 1 mg to about 250 mg, from about 1 mg, to about 150 mg, from about 100 mg, to about 500 mg, or from about 250 mg to about 500 mg of 4SAS.

In order to effectively treat the pathophysiological consequences of the targeted diseases, the administration of the adenylosuccinate, preferably, increases adenosine triphosphate in the subject.

In another example, the adenylosuccinate is administered as a cocktail with at least one additional effective agent. Preferably, the adenylosuccinate is administered as a cocktail with, for example, other pharmaceutically active agents. In still another example, the adenylosuccinate is administered as a food additive as, for example, a granular powder to be placed onto a food item.

### EXAMPLES

The following examples are provided to illustrate the invention, but are not intended to limit the scope thereof.

### Example I

Thirteen (13) young pseudohypertrophic muscular dystrophy patients at various stages across the PMD spectrum, showed increased strength and related functionality while on treatment by administration of 4SAS in intraperitoneal, subcutaneous, and oral dosages. See Table 1.

TABLE 1

| | Se- | Age | Age | Therapy | | |
|---|---|---|---|---|---|---|
| Patient | verity | start | stop | Duration | Dosage | Improved? |
| A | 1 | 2 | 9 | 81-89 | 1 mg-10 mg; | Yes |
| | | | | | 10-20 mg; | |
| | | | | | 100 mg; | |
| | | | | | 200 mg | |
| B | 2 | | | 81-83 | 1 mg-10 mg; | unrelated |
| | | | | | 10-20 mg | death |
| C | 3 | 7.5 | 9.5 | 81-84 | 1 mg-10 mg; | Yes |
| | | | | | 10-20 mg | |
| D | 4 | 8 | 11 | 81-84 | 1 mg-10 mg; | Yes |
| | | | | | 10-20 mg | |
| E | 5 | 12 | 15 | 81-84 | 1 mg-10 mg; | Yes |
| | | | | | 10-20 mg | |
| F | | 9 | 10 | 88-89 | 70 mg | Yes |
| G | | 4 | 6.5 | 87-89 | 150 mg; 333 mg | Yes |
| H | | 6 | 7 | 88-89 | 200 mg; 300 mg | Yes |
| I | | 7 | 10 | 86-89 | 10 mg; 25 mg; | Yes |
| | | | | | 100 mg | |
| J | | 12 | 15 | 86-89 | 1200 mg | Yes |
| K | | 9 | 11 | 87-89 | 200 mg; 300 mg | Yes |
| L | | 6 | 8 | 87-89 | small | Yes |
| M | | 7 | 8 | 88-89 | 100 mg; 300 mg | Yes |
| N | | 4 | 4 | 89-89 | 300 mg | Yes |

### Example II

An ALS patient, previously rendered unable to speak by progression of the disease, temporarily regained capacity to speak intelligibly with her husband, after treatment by administration of 4SAS in an oral dosage.

### Example III

A patient of advanced age (early 70s) experienced increased energy, accelerated healing, and improved posture while on treatment by administration of 4SAS in an oral dosage.

The foregoing description is given for clearness of understanding only, and no unnecessary limitations should be understood therefrom, as modifications within the scope of the invention may be apparent to those having ordinary skill in the art.

1. (canceled)

2. (canceled)

3. A method of treating mitochondrial dysfunction in a subject in need comprising:
administering to the subject an effective amount of a composition comprising an adenylosuccinate (AS)
wherein the adenylosuccinate is a salt of selected from the group consisting of a lithium salt, a sodium salt, a potassium salt, an ammonium salt, a chelated alkali earth metal salt, a chelated transition metal salt, a cationic polymer, and a mixture thereof; and
wherein the adenylosuccinate is a tetra-anion.

4. The method of claim 3, wherein the adenylosuccinate is a tetrasodium adenylosuccinate (4SAS).

5. The method of claim 3, wherein the composition is an oral dosage; and wherein the administration is by oral ingestion.

6. The method of claim 5, wherein the composition is a delayed release formulation that comprises the adenylosuccinate compounded with a delayed release agent.

7. The method of claim 3, wherein the composition is an intravenous dosage; and wherein the administration is sub-cutaneous.

8. The method of claim 3, wherein the administration is trans-dermal.

9. The method of claim 3, wherein the administration of an effective amount treats the pathophysiological consequences of mitochondrial respiratory chain deficiency.

10. A method of treating mitochondrial dysfunction in a subject in need comprising:
administering to the subject an effective amount of a composition comprising an adenylosuccinate (AS);
wherein the administration of the adenylosuccinate increases adenosine triphosphate in the subject.

11. The method of claim 3, wherein the adenylosuccinate is administered as a cocktail with at least one additional effective agent.

12. The method of claim 3, wherein the subject suffers from amyotrophic lateral sclerosis.

13. The method of claim 10, wherein the adenylosuccinate is a salt of selected from the group consisting of a lithium salt, a sodium salt, a potassium salt, an ammonium salt, a chelated alkali earth metal salt, a chelated transition metal salt, a cationic polymer, and a mixture thereof; and wherein the adenylosuccinate is a tetra-anion

14. The method of claim 13, wherein the adenylosuccinate is a tetrasodium adenylosuccinate (4SAS).

15. The method of claim 10, wherein the composition is selected from the group consisting of an oral dosage wherein the administration is by oral ingestion; a delayed release formulation that comprises the adenylosuccinate compounded with a delayed release agent; an intravenous dosage wherein the administration is sub-cutaneous; and a trans-dermal formulation wherein the administration is trans-dermal.

16. The method of claim 10, wherein the administration of an effective amount treats the pathophysiological consequences of mitochondrial respiratory chain deficiency.

17. The method of claim 10, wherein the adenylosuccinate is administered as a cocktail with at least one additional effective agent.

18. The method of claim 10, wherein the subject suffers from amyotrophic lateral sclerosis.
